SEQUENCE LISTING (11 sequences)

SEQ ID NO: 1 - protein, 37 aa, Pseudopleuronectes americanus
  HELIX 1..37
DTASDAAAAAALTAANAKAAAELTAANAAAAAAATAR

SEQ ID NO: 2 - DNA, 111 bp, Pseudopleuronectes americanus
  source 1..111: mol_type = genomic DNA
gacaccgcctctgacgccgccgctgcagccgcccttaccgccgccaacgccaaagccgctgccgaactcactgccgccaacgccgccgccgccgcagcagccaccgccaga

SEQ ID NO: 3 - protein, 469 aa, Escherichia coli
MSAEHVLTMLNEHEVKFVDLRFTDTKGKEQHVTIPAHQVNAEFFEEGKMFDGSSIGGWKGINESDMVLMPDASTAVIDPFFADSTLIIRCDILEPGTLQGYDRDPRSIAKRAEDYLRSTGIADTVLFGPEPEFFLFDDIRFGSSISGSHVAIDDIEGAWNSSTQYEGGNKGHRPAVKGGYFPVPPVDSAQDIRSEMCLVMEQMGLVVEAHHHEVATAGQNEVATRFNTMTKKADEIQIYKYVVHNVAHRFGKTATFMPKPMFGDNGSGMHCHMSLSKNGVNLFAGDKYAGLSEQALYYIGGVIKHAKAINALANPTTNSYKRLVPGYEAPVMLAYSARNRSASIRIPVVSSPKARRIEVRFPDPAANPYLCFAALLMAGLDGIKNKIHPGEAMDKNLYDLPPEEAKEIPQVAGSLEEALNELDLDREFLKAGGVFTDEAIDAYIALRREEDDRVRMTPHPVEFELYYSV

SEQ ID NO: 4 - protein, 12 aa, synthetic construct
  source 1..12: note = linker peptide for linking HPLC6 peptide and the glutamine synthetase
GSGSGSHMGSVD

SEQ ID NO: 5 - DNA, 36 bp, synthetic construct
  source 1..36: mol_type = other DNA; note = nucleotide for expressing linker peptide for linking HPLC6 peptide and the glutamine synthetase
ggtagcggtagcggtagccatatgggatccgtcgac

SEQ ID NO: 6 - DNA, 1410 bp, Escherichia coli
  source 1..1410: mol_type = genomic DNA
atgtccgctgaacacgtactgacgatgctgaacgagcacgaagtgaagtttgttgatttgcgcttcaccgatactaaaggtaaagaacagcacgtcactatccctgctcatcaggtgaatgctgaattcttcgaagaaggcaaaatgtttgacggctcctcgattggcggctggaaaggcattaacgagtccgacatggtgctgatgccagacgcatccaccgcagtgattgacccgttcttcgccgactccaccctgattatccgttcggacatccttgaacctggcaccctgcaaggctatgaccgtgacccgcgctccatgtcgaagcgcgccgaagattacctgcgttccactggcattgccgacaccgtactgttcgggccagaacctgaattcttcctgttcgatgacatccgtttcggatcatctatctccggttcccacgttgctatcgacgatatcgaaggcgcatggaactcctccacccaatacgaaggtggtaacaaaggtcaccgtccggcagtgaaaggcggttacttcccggttccaccggtagactcggctcaggatattcgttctgaaatgtctctggtgatggaacagatgggtctggtggttgaagcccatcaccacgaagtagcgactgctggtcagaacgaagtggctacccgcttcaataccatgaccaaaaaagctgacgaaattcagatctacaaatatgttgtgcacaacgtagcgcaccgcttcggtaaaaccgcgacctttatgccaaaaccgatgttcggtgataacggctccggtatgcactgccacatgtctctgtctaaaaacggcgttaacctgttcgcaggcgacaaatacgcaggtctgtctgagcaggcgctgtactacattggcggcgtaatcaaacacgctaaagcgattaacgccctggcaaacccgaccaccaactcttataagcgtctggtcccgggctatgaagcaccggtaatgctggcttactctgcgcgtaaccgttctgcgtctatccgtattccggtggtttcttctccgaaagcacgtcgtatcgaagtacgtttcccggatccggcagctaacccgtacctgtgctttgctgccctgctgatggccggtcttgatggtatcaagaacaagatccatccgggcgaagccatggacaaaaacctgtatgacctgccgccagaagaagcgaaagagatcccacaggttgcaggctctctggaagaagcactgaacgaactggatctggaccgcgagttcctgaaagccggtggcgtgttcactgacgaagcaattgatgcgtacatcgctctgcgtcgcgaagaagatgaccgcgtgcgtatgactccgcatccggtagagtttgagctgtactacagcgtctaa

SEQ ID NO: 7 - protein, 165 aa, Escherichia coli
MLKPEMIEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDTQN

SEQ ID NO: 8 - protein, 10 aa, synthetic construct
  source 1..10: note = linker peptide for linking HPLC6 peptide and the bacterial non-heme ferritin
GSGSGSHMGS

SEQ ID NO: 9 - DNA, 30 bp, synthetic construct
  source 1..30: mol_type = other DNA; note = nucleotide for expressing linker peptide for linking HPLC6 peptide and the bacterial non-heme ferritin
ggtagcggtagcggtagccatatgggatcc

SEQ ID NO: 10 - DNA, 498 bp, Escherichia coli
  source 1..498: mol_type = genomic DNA
atgctgaaaccagaaatgattgaaaaacttaatgagcagatgaacctggaactgtactcttcactgctttatcagcaaatgagcgcctggtgcagctatcataccttcgaaggtgctgccgcgttcctgcgccgtcacgcccaggaagagatgacgcatatgcagcgtctgtttgattacctgactgataccggcaatttaccgcgtattaataccgttgaatctccgtttgctgaatattcctcacttgatgaattattccaggaaacctataaacacgaacaattaatcacccagaaaattaacgaactggctcatgctgcaatgaccaatcaggactacccaacatttaatttcctgcaatggtatgtttctgagcagcatgaagaagagaaactgttcaaatcgattattgataaattaagcctggcaggcaaaagcggcgaaggtctgtattttatcgacaaagaactctctaccctcgacacacaaaactaa

SEQ ID NO: 11 - protein, 44 aa, synthetic construct
GPHSWEAKLAKALAKALAKHLAKALAKALKACEAGSGSGSHMGS

(19) United States
(12) Patent Application Publication
(10) Pub. No.: US 2026/0338655 A1
(43) Pub. Date: Oct. 29, 2026

(54) CRYO-ELECTRON MICROSCOPE SAMPLE PREPARATION KIT AND USE OF SAME

(71) Applicant: Inter-University Research Institute Corporation High Energy Accelerator Research Organization, Tsukuba-shi (JP)

(72) Inventors: Masato Kawasaki, Tsukuba-shi (JP); Toshio Moriya, Tsukuba-shi (JP)

(21) Appl. No.: 19/140,010

(22) PCT Filed: Dec. 20, 2023
(86) PCT No.: PCT/JP2023/045749

§ 371 Date: Jun. 17, 2025

(30) Foreign Application Priority Data
Dec. 21, 2022 (JP) 2022-204279

(51) Int. Cl. G01N 1/42; C07K 14/00; C07K 14/46; C12N 9/00; C12N 9/02

(52) U.S. Cl. CPC G01N 1/42; C07K 14/001; C07K 14/461; C12N 9/0091; C12N 9/93; C12Y 116/03001; C12Y 603/01002

(57) ABSTRACT

The cryo-electron microscope sample preparation kit contains an amphipathic protein and a protein crosslinking agent, or an expression vector containing a nucleic acid encoding amphipathic protein. The cryo-electron microscope sample preparation kit contains an ice-thickness controlling molecule to which an amphipathic protein is bonded. The amphipathic protein may have an amphipathic α-helix structure. The amphipathic protein may be an antifreeze protein or a type I antifreeze protein.

The cryo-electron microscope sample preparation kit contains an amphipathic protein and a protein crosslinking agent, or an expression vector containing a nucleic acid encoding amphipathic protein. The cryo-electron microscope sample preparation kit contains an ice-thickness controlling molecule to which an amphipathic protein is bonded. The amphipathic protein may have an amphipathic α-helix structure. The amphipathic protein may be an antifreeze protein or a type I antifreeze protein.

### TECHNICAL FIELD

The present invention relates to a cryo-electron microscope sample preparation kit and use thereof. Specifically, the present invention relates to a cryo-electron microscope sample preparation kit, a preparation method for a cryo-electron microscope sample, a method for controlling the posture of a target protein in a cryo-electron microscope sample, a method for suppressing collision and adsorption of a target protein in a cryo-electron microscope sample onto an amorphous carbon film, and a method for controlling the thickness of ice in a cryo-electron microscope sample.

Priority is claimed on Japanese Patent Application No. 2022-204279, filed Dec. 21, 2022, the content of which is incorporated herein by reference.

### BACKGROUND ART

In the related art, an X-ray crystallography method, an NMR method, and the like have been used as a method of analyzing the structure of a biological molecule such as a protein. However, in recent years, single particle analysis using a cryo-electron microscope (hereinafter, simply referred to as a “cryo-electron microscope method”) has attracted attention. In the cryo-electron microscope method, crystallization of a sample, which is essential for X-ray crystal structure analysis, is not required, and there is no upper limit on the molecular weight of the sample, which restricts the application of the NMR method.

However, since most of the interatomic bonds that maintain the three-dimensional structure of the biological molecule are non-covalent bonds such as hydrogen bonds or ionic bonds, the biological molecule is more likely to be damaged by electron beam irradiation as compared with a sample such as a metal or a semiconductor. Therefore, in the cryo-electron microscope method, to record a high-resolution projection image (hereinafter, referred to as a “particle image”) of a biological molecular particle with high image quality while suppressing damage as much as possible, it is required to embed a biological molecule in an amorphous ice thin film by rapidly freezing an aqueous solution of the biological molecule as a thin film, irradiate the biological molecule with an electron beam while maintaining the biological molecule at a low temperature on a sample stage cooled with liquid helium or liquid nitrogen, and record an electron microscope image. In addition, depending on the type of the biological molecule, the biological molecule may be adsorbed on the amorphous carbon film of the sample grid and may not be dispersed in the entire hole in which the amorphous ice thin film, which is the observation region, is formed.

Furthermore, since each particle image is a projection image of a molecule embedded in the ice thin film in various orientations, it is also necessary to collect as many images as possible, which are projected in various directions, as uniformly as possible to reconstruct the three-dimensional image. That is, it is essential to reduce the noise level and improve the signal level by efficiently collecting as many particle images as possible, aligning the orientation and position of the particle images, and classifying and averaging the particle images for each projection direction. Then, the relative relationship in the projection direction of the particle images is determined and back-projected, whereby it is possible to reconstruct the three-dimensional image with high resolution for the first time. However, in some cases, only a projection image of the particles that have been partially damaged due to the collision with the gas-liquid interface or a projection image obtained by imaging the particles from a specific direction because of the adsorption onto the gas-liquid interface can be obtained.

On the other hand, the antifreeze protein, which is an amphipathic protein, is a relatively small protein consisting of a polypeptide of about 30 or more amino acid residues and 150 or less amino acid residues, and it is known that the antifreeze protein has an ability to inhibit the freezing of water or a water-containing substance by being bound to a specific crystal plane of an ice nucleus and suppressing the growth of the ice nucleus in a temperature range of 0 °C or lower. In recent years, it has been revealed that the antifreeze protein has a property of accumulating at a gas-liquid interface (see, for example, Non-Patent Document 1 or the like).

### CITATION LIST

### Non-Patent Document

Non-Patent Document 1: Meister K et al., “Investigation of the Ice-Binding Site of an Insect Antifreeze Protein Using Sum-Frequency Generation Spectroscopy.”, J. Phys. Chem. Lett., Vol. 6, Issue 7, pp. 1162-1167, 2015.

### SUMMARY OF INVENTION

### Technical Problem

The present invention has been made in view of the above circumstances, and provides a cryo-electron microscope sample preparation kit, which makes it possible to obtain a cryo-electron microscope sample, in which a bias of an undesirable posture of the target protein in a sample with respect to an electron beam incidence direction and a collision with and adsorption onto a gas-liquid interface are suppressed, and in which the target protein is dispersed over an entire observation region. In addition, there is provided a cryo-electron microscope sample preparation kit, which makes it possible to obtain a cryo-electron microscope sample in which the thickness of ice of a sample is uniformly controlled. Furthermore, there is provided a preparation method for a cryo-electron microscope sample, a method for controlling the posture of a target protein in a cryo-electron microscope sample, a method for suppressing adsorption of a target protein in a cryo-electron microscope sample onto an amorphous carbon film, a method for suppressing collision and adsorption of a target protein in a cryo-electron microscope sample onto a gas-liquid interface, and a method for controlling the thickness of ice in a cryo-electron microscope sample, which use the cryo-electron microscope sample preparation kit.

### Solution to Problem

That is, the present invention includes the following aspects.
(1) A cryo-electron microscope sample preparation kit, containing an amphipathic protein or an expression vector containing a nucleic acid encoding the amphipathic protein.
(2) A cryo-electron microscope sample preparation kit, containing an ice-thickness controlling molecule to which an amphipathic protein is bonded.
(3) The cryo-electron microscope sample preparation kit according to (1) or (2), in which the amphipathic protein has an amphipathic α-helix structure.
(4) The cryo-electron microscope sample preparation kit according to any one of (1) to (3), in which the amphipathic protein is an antifreeze protein.
(5) The cryo-electron microscope sample preparation kit according to (4), in which the antifreeze protein is a type I antifreeze protein.
(6) The cryo-electron microscope sample preparation kit according to (5), in which the type I antifreeze protein is an HPLC6 peptide.
(7) The cryo-electron microscope sample preparation kit according to any one of (1) to (3), in which the amphipathic protein is a KALA peptide.
(8) The cryo-electron microscope sample preparation kit according to (1), further containing a protein crosslinking agent.
(9) The cryo-electron microscope sample preparation kit according to (8), in which the protein crosslinking agent is an amine-reactive crosslinking agent.
(10) The cryo-electron microscope sample preparation kit according to (9), in which the amine-reactive crosslinking agent is glutaraldehyde, or bis(3-sulfo-N-succinimidyl)suberate or a salt thereof.
(11) The cryo-electron microscope sample preparation kit according to (1), further containing an ice-thickness controlling molecule to which an amphipathic protein is bonded.
(12) The cryo-electron microscope sample preparation kit according to (2) or (11), in which the ice-thickness controlling molecule is an antifreeze protein and is a homo-multimeric protein.
(13) The cryo-electron microscope sample preparation kit according to (12), in which the homo-multimeric protein is a glutamine synthetase or ferritin.
(14) A preparation method for a cryo-electron microscope sample, including preparing a cryo-electron microscope sample containing a target protein using the cryo-electron microscope sample preparation kit according to any one of (1) to (13).
(15) A method for controlling the posture of a target protein in a cryo-electron microscope sample, the method including preparing a cryo-electron microscope sample containing a target protein using the cryo-electron microscope sample preparation kit according to any one of (1), (8) to (11).
(16) A method for suppressing adsorption of a target protein in a cryo-electron microscope sample onto an amorphous carbon film, the method including preparing a cryo-electron microscope sample containing a target protein using the cryo-electron microscope sample preparation kit according to any one of (1), (8) to (11).
(17) A method for suppressing collision and adsorption of a target protein in a cryo-electron microscope sample onto a gas-liquid interface, the method including preparing a cryo-electron microscope sample containing a target protein using the cryo-electron microscope sample preparation kit according to any one of (1), (8) to (11).
(18) A method for controlling the thickness of ice in a cryo-electron microscope sample, the method including preparing a cryo-electron microscope sample containing a target protein using the cryo-electron microscope sample preparation kit according to any one of (2), (11) to (13).

### Advantageous Effects of Invention

According to the cryo-electron microscope sample of the above-described aspect, it is possible to obtain a cryo-electron microscope sample, in which a bias of an undesirable posture of the target protein in a sample with respect to an electron beam incidence direction and a collision with and adsorption onto a gas-liquid interface are suppressed, and in which the target protein is dispersed over an entire observation region. In addition, according to the cryo-electron microscope sample of the above-described aspect, it is possible to obtain a cryo-electron microscope sample in which the thickness of ice of a sample is uniformly controlled.

### BRIEF DESCRIPTION OF DRAWINGS

FIG. 1 A two-dimensional class average image of a particle image of a hemagglutinin protein to which an HPLC6 peptide is bonded, which is captured by a cryo-electron microscope in Example 1.

FIG. 2A An image of glutamine synthetase, which is captured by a cryo-electron microscope in Example 2.

FIG. 2B An image of a glutamine synthetase to which an HPLC6 peptide is bonded, which is captured by a cryo-electron microscope in Example 2.

FIG. 3 An image of ferritin to which HPLC6 peptide is bonded, which is captured by a cryo-electron microscope in Example 3.

FIG. 4 An image of a mixed solution of a hemagglutinin protein and ferritin to which an HPLC6 peptide are bonded, which is captured by a cryo-electron microscope in Example 4.

FIG. 5 A two-dimensional class average image of a particle image of inosine-5′-monophosphate dehydrogenase 2 (IMPDH2), which is captured by a cryo-electron microscope in Example 5.

FIG. 6A An image of ferritin, which is captured by a cryo-electron microscope in Example 6.

FIG. 6B An image of a fusion protein of ferritin and a KALA peptide, which is captured by a cryo-electron microscope in Example 6.

### DESCRIPTION OF EMBODIMENTS

### <<Cryo-Electron Microscope Sample Preparation Kit>>

### First Embodiment

The cryo-electron microscope sample preparation kit according to one embodiment of the present invention (hereinafter, may be referred to as a “preparation kit of the present embodiment”) includes an antifreeze protein or an expression vector containing a nucleic acid encoding the antifreeze protein.

In the cryo-electron microscope method, to reconstruct a three-dimensional image of a target protein, it is necessary to collect as many images of the target protein as possible, which are projected in various directions as uniform as possible. However, in the sample grid for a cryo-electron microscope, a bias in the posture of the protein with respect to the electron beam incidence direction may occur, and particle images having the same orientation may account for the majority. In addition, the protein may be adsorbed to an amorphous carbon film having holes (diameter of about 1 μm) as an observation region of the grid, and may not be uniformly dispersed inside the observation region, and thus a sufficient number of particle images may not be obtained. Furthermore, in some cases, only a projection image of the particles that have been partially damaged due to the collision with the gas-liquid interface or a projection image obtained by imaging the particles from a specific direction because of the adsorption onto the gas-liquid interface can be obtained.

On the other hand, in the preparation kit of the present embodiment, because of the property that the antifreeze protein accumulates at the gas-liquid interface, the target protein, which has a property of being easily adsorbed to the gas-liquid interface in a specific posture, is mixed with the antifreeze protein to protect the gas-liquid interface, and the target protein can be suppressed from colliding with or adsorbing to the gas-liquid interface. Accordingly, it is possible to reduce the particle image of the target protein that has been partially damaged and to reduce and eliminate the preferred orientation of the target protein particles. That is, it is possible to suppress the bias of the posture of the protein with respect to the electron beam incidence direction and to acquire the projection image of the protein having various postures.

In addition, because of the property that the antifreeze protein accumulates at the gas-liquid interface, the preparation kit of the present embodiment can suppress the adsorption of the target protein onto the amorphous carbon film and can uniformly disperse the target protein inside the observation region (hole) of the grid.

It is noted that in the present specification, the cryo-electron microscope method means a method in which a sample solution of a biological molecule such as an isolated and purified protein is rapidly frozen to embed the biological molecule in a thin ice film, a few hundred to a few thousand images are acquired using a special transmission electron microscope (TEM) that can perform observation by irradiating the sample with an electron beam while maintaining the sample at a low temperature under cooling with liquid nitrogen (approximately −196 °C), and a three-dimensional structure of a target biological molecule is reconstructed from the acquired electron microscope image by computer image processing.

In addition, in the present specification, the cryo-electron microscope sample is a sample produced by rapidly freezing a solution containing a target protein on a film hole grid. A film hole grid used in a cryo-electron microscope has a structure in which a support film having regularly opened holes is further attached onto a metal mesh that has holes in a lattice shape and is composed of a conductive material such as copper, gold, or molybdenum. A thin carbon film of about 10 nm or more and about 50 nm or less is usually used as the support film, but the thin carbon film coated with a thin gold film of about 5 nm or more and about 10 nm or less is also used to prevent the film from being charged by electron beam irradiation.

### [Antifreeze Protein]

The antifreeze protein is a protein found in fish and the like that live in a low temperature environment, and is a protein that lowers a solidifying point (temperature at the time of freezing) in a living body and contributes to the maintenance of life of the organism by preventing freezing and recrystallization of ice crystals. The antifreeze protein strongly binds to the surface of innumerable small ice crystals generated inside water at the moment of freezing, and suppresses the growth of the ice crystals.

The antifreeze proteins derived from fish are classified into four types according to their structures. The type I antifreeze protein is an α-helix type protein that contains a large number of alanine residues and has threonine residues and aspartic acid residues arranged at regular intervals. The type II antifreeze protein is a C-type lectin-like protein containing a disulfide bond. The type III antifreeze protein is a spherical protein consisting of a characteristic structural motif. The type IV antifreeze protein is a protein containing a large number of glutamine residues and having an unknown three-dimensional structure. Among these, the type I antifreeze protein is preferable since the crystal structure is known, the molecular weight is small, the structure is simple, and the type I antifreeze protein does not interfere with the projection of the particle image of the target protein. Examples of the preferred type I antifreeze protein include an HPLC6 peptide consisting of 37 amino acid residues (amino acid sequence: SEQ ID NO: 1), and the like. The HPLC6 peptide represented by SEQ ID NO: 1 has a partial amino acid sequence of an antifreeze protein derived from Pseudopleuronectes americanus. The full-length amino acid sequence of the antifreeze protein derived from Pseudopleuronectes americanus is described in, for example, GenBank accession numbers AB59964.1, CAA30389.1, AAA49469.1, AAA49471.1, AAA49472.1, and the like.

In addition, in the preparation kit of the present embodiment, in a case where an expression vector containing a nucleic acid encoding an antifreeze protein is used, the antifreeze protein can be bonded to any position of the target protein by inserting the nucleic acid encoding the target protein upstream or downstream of the nucleic acid encoding the antifreeze protein. Accordingly, because of the property that the antifreeze protein accumulates at the gas-liquid interface, the surface of the target protein to which the antifreeze protein is bonded faces the gas-liquid interface, and thus the bias of the posture of the protein with respect to the electron beam incidence direction is suppressed, and the projection image of the protein in various postures can be acquired.

### [Protein Crosslinking Agent]

Alternatively, in the preparation kit of the present embodiment, the antifreeze protein can be bonded to any position of the target protein by using the antifreeze protein and the protein crosslinking agent in combination.

As the protein crosslinking agent, a crosslinking agent generally used for crosslinking proteins can be used. Specific examples of the protein crosslinking agent include an amine-reactive crosslinking agent that crosslinks between primary amino groups (—NH₂), a sulfhydryl-reactive crosslinking agent that crosslinks between sulfhydryl groups, an amino group-sulfhydryl group crosslinking agent that crosslinks between an amino group and a sulfhydryl group, a carboxy group-amino group crosslinking agent that crosslinks between a carboxy group and an amino group, and a hydroxy group-sulfhydryl group crosslinking agent that crosslinks between a hydroxy group and a sulfhydryl group.

The primary amino group is present at the N-terminal or the side chain of lysine of the target protein or the antifreeze protein and is positively charged at a physiological pH. Therefore, the primary amino group is mainly present on the outer surface of the three-dimensional structure of the target protein. Therefore, the amine-reactive crosslinking agent is preferable as the protein crosslinking agent because of being suitable for crosslinking the primary amino groups present on the outer surface.

Examples of the amine-reactive crosslinking agent include a crosslinking agent having an N-hydroxyester (NHS ester) group as a reaction group, such as N,N′-disuccinimidyl glutarate (DSG), disuccinimidyl suberate (DSS), bis(3-sulfo-N-succinimidyl)suberate (BS3), and salts thereof, a crosslinking agent having an imide ester group as a reaction group, such as dimethyl adipimidate (DMA), dimethyl pimelimidate (DMP), and dimethyl suberimidate (DMS); a crosslinking agent having an aldehyde group as a reaction group, such as formaldehyde and glutaraldehyde; and the like.

Among these, as the amine-reactive crosslinking agent, glutaraldehyde or bis(3-sulfo-N-succinimidyl)suberate, or a salt thereof is preferable, and bis(3-sulfo-N-succinimidyl)suberate or a salt thereof is more preferable since a polymerization reaction between crosslinking agents does not occur.

### [Expression Vector]

The preparation kit of the present embodiment can include an expression vector containing a nucleic acid encoding an antifreeze protein, instead of the antifreeze protein and the protein crosslinking agent, or in addition to the antifreeze protein and the protein crosslinking agent. Examples of the antifreeze protein include the same proteins as exemplified in the above-described “antifreeze protein”. Examples of the nucleic acid encoding the antifreeze protein include a nucleic acid encoding an HPLC6 peptide, and the like. The nucleic acid (SEQ ID NO: 2) encoding the HPLC6 peptide is a partial base sequence of a nucleic acid encoding an antifreeze protein derived from Pseudopleuronectes americanus. The full-length base sequence of the antifreeze protein derived from Pseudopleuronectes americanus is described in, for example, GenBank accession numbers AH005322, X07506, M62414, M62416, M62417, and the like.

The expression vector into which the above-described nucleic acid is inserted is not particularly limited, and for example, a plasmid derived from Escherichia coli, a plasmid derived from Bacillus subtilis, a yeast-derived plasmid, a bacteriophage, a virus vector, a vector obtained by modifying these vectors, and the like can be used. Examples of the plasmid derived from Escherichia coli include pBR322, pBR325, pUC12, pUC13, and the like. Examples of the plasmid derived from Bacillus subtilis include pUB110, pTP5, pC194, and the like. Examples of the yeast-derived plasmid include pSH19, pSH15, and the like. Examples of the bacteriophage include a λ phage and the like. Examples of the virus from which the virus vector is derived include adenovirus, adeno-associated virus, lentivirus, vaccinia virus, baculovirus, retrovirus, hepatitis virus, and the like.

In the expression vector, the promoter for expressing the antifreeze protein is not particularly limited, and may be a promoter for expression using animal cells as a host, may be a promoter for expression using plant cells as a host, or may be a promoter for expression using insect cells as a host. Examples of the promoter for expression using animal cells as a host include an EF1α promoter, an SRα promoter, an SV40 promoter, an LTR promoter, a cytomegalovirus (CMV) promoter, an HSV-tk promoter, a CAG promoter, and the like. Examples of the promoter for expression using plant cells as a host include a 35S promoter of cauliflower mosaic virus (CaMV), a rubber elongation factor (REF) promoter, and the like. Examples of the promoter for expression using insect cells as a host include a polyhedrin promoter, a p10 promoter, and the like. These promoters can be appropriately selected depending on the type of the host that expresses the antifreeze protein.

The expression vector may further have a multi-cloning site, an enhancer, a splicing signal, a poly(A) addition signal, a selection marker, a replication origin, or the like.

In the expression vector, it is preferable to add an individual target gene (in the present embodiment, a gene encoding a target protein) to the upstream or downstream of the nucleic acid encoding the antifreeze protein. Alternatively, in the expression vector, the nucleic acid encoding the antifreeze protein may be inserted into the middle of the base sequence of the gene encoding the target protein. In this case, by appropriately adjusting the positional relationship between the nucleic acid encoding the antifreeze protein in the expression vector and the gene encoding the target protein, it is possible to prepare a fusion protein in which the antifreeze protein is bonded to a desired position of the target protein. That is, the insertion position of the nucleic acid encoding the antifreeze protein can be appropriately selected according to the secondary structure of the target protein, and for example, the insertion position can be set such that the antifreeze protein is inserted into a loop portion connecting the α-helix structure and the β-sheet.

As described above, the expression vector includes a nucleic acid encoding an antifreeze protein and a gene encoding a target protein, whereby a fusion protein in which the antifreeze protein is fused with the target protein can be produced. In addition, in a case of being incorporated into an expression vector having a protein secretion type signal to the outside of the cell, a fusion protein of the secretion type signal and the target protein in which an amino acid sequence of the antifreeze protein is added can be produced in a culture solution and recovered. In addition, the same antifreeze protein-added fusion protein can be produced even in the case of the intracellular expression type vector.

As described above, a fusion protein of the above-described antifreeze protein and the target protein can be expressed by using an expression vector into which a nucleic acid encoding an antifreeze protein and a gene encoding a target protein are inserted, and an appropriate host cell according to the type of the expression vector.

### Second Embodiment

The cryo-electron microscope sample preparation kit of one embodiment of the present invention (hereinafter, may be referred to as a “preparation kit of the present embodiment”) includes an ice-thickness controlling molecule to which the antifreeze protein is bonded.

In the cryo-electron microscope method, to suppress damage as much as possible and record a high-resolution particle image with high image quality, it is necessary to rapidly freeze an aqueous solution of the target protein as a uniform thin film to embed the target protein in an amorphous thin ice film. However, depending on the type of the target protein, the target protein is aggregated concentrating in the central portion of the observation region of the grid or aggregated at the edge of the observation region, whereby the thickness of the ice may not be uniform and the ice may be formed to be thick.

On the other hand, according to the preparation kit of the present embodiment, the ice-thickness controlling molecules to which the antifreeze protein is bonded and which have a certain thickness or a certain particle diameter are uniformly dispersed inside the observation region (hole) of the grid because of the property that the antifreeze protein accumulates at the gas-liquid interface, whereby it is possible to form uniform ice with a thickness of several tens of nm depending on the thickness or the particle diameter of the ice-thickness controlling molecule.

### [Ice-Thickness Controlling Molecule]

As the ice-thickness controlling molecule, a polymer satisfying the following requirements can be used.
1) The particle diameter of the ice-thickness controlling molecule is slightly larger than the particle diameter of the target protein.
2) The shape of the ice-thickness controlling molecule is different from the shape of the target protein, which makes it easy to distinguish.
3) The protein can bind to an antifreeze protein.

Specific examples of such an ice-thickness controlling molecule include a homo- or hetero-multimeric protein and the like, but the present invention is not limited thereto.

Among these, as the ice-thickness controlling molecule, a homo-multimeric protein can be preferably used.

The homo-multimeric protein is not particularly limited as long as it is a protein having a known crystal structure and in which a monomer self-assembles in a solution to form a multimer. The thickness or particle diameter of the homo-multimeric protein are only required to be the same size as the desired thickness of the ice.

Specific examples of the homo-multimeric protein include glutamine synthetase (a double-ring-like structure, a diameter of about 16 nm, a thickness of about 11 nm), ferritin (spherical, a particle diameter of about 14 nm), and the like.

These homo-multimeric proteins are obtained by expressing a monomer constituting the homo-multimeric protein and allowing the monomer to self-assemble in a solution. The antifreeze protein may be bonded to the homo-multimeric protein using the above-described protein crosslinking agent, or may be bonded by expressing a fusion protein of a monomer and the antifreeze protein. Among these, it is preferable to allow a fusion protein of a monomer and an antifreeze protein to be expressed to bind the antifreeze protein to the ice-thickness controlling molecule since the binding position of the antifreeze protein is easily adjusted.

The fusion protein of a monomer and an antifreeze protein can be expressed, for example, by transforming an appropriate host cell with an expression vector containing a nucleic acid encoding the monomer and a nucleic acid encoding the antifreeze protein, according to the type of the expression vector. The type of the expression vector and the insertion position of the nucleic acid encoding a monomer and the nucleic acid encoding an antifreeze protein in the expression vector are as described in the preparation kit of the first embodiment.

In this case, the antifreeze protein may be bonded to all the monomers, or may be bonded to a part of the monomers. That is, a homo-multimeric protein in which the antifreeze protein is bonded to all of the monomers may be prepared by self-assembly of the fusion protein of the monomer and the antifreeze protein in a solution, or a homo-multimeric protein in which the antifreeze protein is bonded to a part of the monomers may be prepared by self-assembly of the fusion protein of the monomer and the antifreeze protein, and only monomer in a solution.

Among these, it is preferable that the antifreeze protein is bonded to all the monomers since the homo-multimeric protein can be dispersed in the sample in a form in which the thickness or the particle diameter is uniform by more efficiently directing the desired surface of the homo-multimeric protein toward the gas-liquid interface. That is, the preparation kit of the present embodiment can also include an expression vector containing a nucleic acid encoding a monomer constituting the homo-multimeric protein and a nucleic acid encoding the antifreeze protein (or a nucleic acid encoding a fusion protein of the monomer constituting the homo-multimeric protein and the antifreeze protein) instead of the homo-multimeric protein to which the antifreeze protein is bonded.

Specific examples of the monomer constituting the homo-multimeric protein include a monomer constituting glutamine synthetase (see amino acid sequence: for example, GenBank accession numbers CAA28806.1, AAA23879.1, AAB03004.1, AAC76867.1, BAE77439.1, AAA98066.1, AAA23882.1, and AAA23880.1, and see base sequence: for example, GenBank accession numbers X05173, M13746, L19201, U00096, AP009048, J01618, M10421, and K02176), a monomer constituting ferritin (see amino acid sequence: for example, GenBank accession numbers CAA37593.1, AAC74975.1, BAA15728.1, and AAA79049.1, and see base sequence: for example, GenBank accession numbers X53513, U00096, AP009048, and U35066), and the like.

In addition, examples of the nucleic acid encoding the fusion protein of a monomer constituting a homo-multimeric protein and an antifreeze protein include a nucleic acid encoding a fusion protein as an antifreeze protein, in which an HPLC6 peptide, which is a type I antifreeze protein, is bonded to the N-terminal of a monomer of glutamine synthetase, a nucleic acid encoding a fusion protein in which an HPLC6 peptide as an antifreeze protein, which is a type I antifreeze protein, is bonded to the N-terminal of a monomer of ferritin, and the like.

### <Other Configurations>

The preparation kit of the present embodiment can further include a host cell depending on the type of the expression vector, a known buffer solution (such as phosphate buffered saline (PBS)) for dissolving or dispersing the target protein, a film hole grid generally used for a cryo-electron microscope sample, and the like, in addition to the above-described configuration.

In addition, as the preparation kit of the present embodiment, the cryo-electron microscope sample preparation kit according to the first embodiment and the cryo-electron microscope sample preparation kit according to the second embodiment may be used in combination.

That is, the preparation kit according to the first embodiment of the present invention may further include a homo-multimeric protein to which the antifreeze protein is bonded.

Alternatively, the preparation kit according to the second embodiment of the present invention may further include an expression vector containing an antifreeze protein and a protein crosslinking agent, or a nucleic acid encoding an antifreeze protein.

Accordingly, it is possible to solve three issues of the bias of the posture of the target protein with respect to the electron beam incidence direction, the localization of the target protein such as adsorption onto the amorphous carbon film, and the control of the thickness of the ice in the cryo-electron microscope sample. That is, by using the cryo-electron microscope sample preparation kit according to the first embodiment and the cryo-electron microscope sample preparation kit according to the second embodiment in combination, a cryo-electron microscope sample in which a bias of the posture of a target protein in the sample with respect to an electron beam incident direction is suppressed, the target protein is dispersed over the entire observation region, and the thickness of ice of the sample is uniformly controlled can be obtained.

<<Preparation method for cryo-electron microscope sample>>

The preparation method for a cryo-electron microscope sample according to one embodiment of the present invention (hereinafter, may be referred to as a “preparation method of the present embodiment”) includes preparing a cryo-electron microscope sample containing a target protein (hereinafter, may be referred to as a “preparation step”) using the cryo-electron microscope sample preparation kit according to the first embodiment or the second embodiment.

According to the preparation method of the present embodiment, mainly, by using the cryo-electron microscope sample preparation kit according to the first embodiment, it is possible to suppress the bias of the posture of the target protein in the sample with respect to the electron beam incidence direction. That is, the preparation method of the present embodiment can also be said to be a method for controlling the posture of a target protein in a cryo-electron microscope sample. The same applies to a case where the preparation kit of the first embodiment further includes an ice-thickness controlling molecule to which an amphipathic protein is bonded.

According to the preparation method of the present embodiment, mainly, by using the cryo-electron microscope sample preparation kit according to the first embodiment, it is possible to suppress the target protein from colliding with or adsorbing to the gas-liquid interface. That is, the preparation method of the present embodiment can also be said to be a method for suppressing adsorption of a target protein in a cryo-electron microscope sample onto a gas-liquid interface, or a method for suppressing collision of a target protein in a cryo-electron microscope sample on a gas-liquid interface. The same applies to a case where the preparation kit of the first embodiment further includes an ice-thickness controlling molecule to which an amphipathic protein is bonded.

According to the preparation method of the present embodiment, mainly, by using the cryo-electron microscope sample preparation kit according to the first embodiment, it is possible to suppress the adsorption of the target protein onto the amorphous carbon film in the sample, and to disperse the target protein over the entire observation region. That is, the preparation method of the present embodiment can also be said to be a method for suppressing adsorption of a target protein in a cryo-electron microscope sample onto an amorphous carbon film. The same applies to a case where the preparation kit of the first embodiment further includes an ice-thickness controlling molecule to which an amphipathic protein is bonded.

According to the preparation method of the present embodiment, mainly, by using the cryo-electron microscope sample preparation kit according to the second embodiment, it is possible to control the thickness of ice of the sample. That is, the preparation method of the present embodiment can also be said to be a method for controlling the thickness of ice in a cryo-electron microscope sample.

Next, the steps constituting the preparation method of the present embodiment will be described in detail.

### <Preparation Step>

In the preparation step, a cryo-electron microscope sample containing a target protein is prepared using the cryo-electron microscope sample preparation kit according to the first embodiment or the second embodiment.

Specifically, for example, in a case where the antifreeze protein is used as the preparation kit of the first embodiment, the antifreeze protein and the target protein are mixed in a buffer solution having a pH of 7 to 9.

In this case, the mixing ratio of the target protein and the antifreeze protein may be 1:1 to 1:5 or may be 1:1 to 1:2 in terms of molar ratio. By setting the molar ratio within the above-described range, it is possible to suppress the target protein from colliding with or adsorbing to the gas-liquid interface. As a result, the bias of the posture of the target protein with respect to the electron beam incidence direction can be further suppressed and the target protein can be projected in various postures.

In addition, for example, in a case where the protein crosslinking agent and the antifreeze protein are used in combination as the preparation kit of the first embodiment, first, the target protein and the antifreeze protein are mixed in a solution in the presence of the protein crosslinking agent to bind the antifreeze protein to the target protein.

In this case, the mixing ratio of the target protein and the antifreeze protein may be 1:10 to 1:200 or may be 1:20 to 1:120 in terms of molar ratio. By setting the molar ratio within the above-described range, it is possible to sufficiently bind the antifreeze protein to the target protein. As a result, the bias of the posture of the target protein with respect to the electron beam incidence direction can be further suppressed and the target protein can be projected in various postures.

In addition, the protein crosslinking agent can be used according to a known protocol depending on the type thereof. For example, in the case of an amine-reactive crosslinking agent, the crosslinking agent is used at a concentration of about 10 times molar or more and about 50 times molar or less with respect to the total molar amount of the protein in a buffer solution that does not contain an amine having a pH of 7 to 9. The amine-reactive crosslinking agent is used in a solution at a specific concentration of about 0.25 mM to 15.00 mM. The crosslinking reaction is performed at room temperature (about 25 °C) for, for example, 1 minute or more and 60 minutes or less, or on ice for 1 hour or more and 5 hours or less. After the reaction, the crosslinking reaction is stopped by adding a solution containing a reaction terminator or removing an unreacted amine-reactive crosslinking agent by dialysis or desalting.

In addition, for example, in a case where an expression vector containing a nucleic acid encoding an antifreeze protein is used as the preparation kit of the first embodiment, a gene encoding a target protein is inserted at a desired position of the expression vector. The insertion position of the nucleic acid encoding the antifreeze protein and the gene encoding the target protein in the expression vector is as described in the preparation kit of the first embodiment. Next, a fusion protein of an antifreeze protein and a target protein is allowed to be expressed by transforming an appropriate host cell with the expression vector containing a nucleic acid encoding the antifreeze protein and a gene encoding the target protein, according to the type of the expression vector. The fusion protein is obtained from a culture solution of a host cell or from an inside of the host cell by lysing the host cell. The obtained fusion protein may be purified by a known method, as necessary.

Alternatively, for example, in a case where the preparation kit of the second embodiment is used, the homo-multimeric protein to which the antifreeze protein is bonded and the target protein are mixed in a buffer solution containing no amine having a pH of 7 or more and 9 or less. The target protein may be used as it is, or a target protein to which the antifreeze protein is bonded may be used by using the preparation kit of the first embodiment.

The concentration of the homo-multimeric protein to which the antifreeze protein is bonded and the concentration of the target protein, which are contained in the mixed solution of the homo-multimeric protein to which the antifreeze protein is bonded and the target protein can be appropriately adjusted depending on the type of the homo-multimeric protein to be used. For example, in a case where the homo-multimeric protein is ferritin, the concentration may be 0.1 μM or more and 3.0 μM or less, or may be 0.5 μM or more and 1.5 μM or less. In addition, at this time, the concentration of the target protein contained in the mixed solution may be 1.0 μM or more and 5.0 μM or less, or may be 2.0 μM or more and 4.0 μM or less. In a case where the concentration of these proteins is within the above-described range, the existing amount of the homo-multimeric protein to which the antifreeze protein is bonded can be set to an amount of a degree not interfering with the projection of the target protein while forming uniform ice according to the thickness or the particle diameter of the homo-multimeric protein.

Next, the solution containing the target protein to which the antifreeze protein is bonded, or the solution containing the homo-multimeric protein in which the target protein and the antifreeze protein are bonded to each other, which is obtained as described above, is placed on the film hole grid subjected to the hydrophilization treatment by glow discharge or the like. Next, after the excess solution is removed using filter paper or the like, the grid is rapidly dropped into liquid ethane to freeze the sample. Accordingly, a frozen grid in which the target protein is trapped in a thin amorphous ice film is obtained. The obtained frozen grid is stored in liquid nitrogen until observation with a cryo-electron microscope.

The cryo-electron microscope sample obtained by the preparation method of the present embodiment is attached to a cryo transfer holder or a dedicated cartridge in a cryo workstation cooled with liquid nitrogen, and is inserted into a cryo-electron microscope without being frosted, and imaged. It is possible to perform the structural analysis of the target protein by single particle analysis based on the captured data. In addition, the present invention can also be applied to structural analysis using sub-tomogram averaging using electron tomography.

Although each embodiment using the antifreeze protein (or the vector containing the nucleic acid encoding the antifreeze protein) has been described above, the preparation kit of each of the above-described embodiments may include an amphipathic protein (or a vector containing a nucleic acid encoding an amphipathic protein) other than the antifreeze protein instead of the antifreeze protein (or the vector containing the nucleic acid encoding the antifreeze protein) or together with the antifreeze protein.

In addition, in the preparation method of each of the above-described embodiments, an amphipathic protein other than the antifreeze protein (or a vector containing a nucleic acid encoding the amphipathic protein) may be used instead of the antifreeze protein (or the vector containing a nucleic acid encoding the antifreeze protein) or together with the antifreeze protein.

The amphipathic protein has a portion rich in hydrophobicity and a portion rich in hydrophilicity. Accordingly, since the portion rich in hydrophobicity of the amphipathic protein faces air and a portion rich in hydrophilicity faces water, the amphipathic protein has properties of being accumulated at a gas-liquid interface, not limited to the antifreeze protein. Therefore, the same effect can be obtained even in a case where an amphipathic protein other than the antifreeze protein is used.

As the amphipathic protein, an amphipathic protein having a small and relatively simple structure is preferable, and an amphipathic protein having an amphipathic α-helix structure is particularly preferable. Examples of such a protein include a type I antifreeze protein such as HPLC6 shown in Examples, a KALA peptide, or the like, but the present invention is not limited thereto.

### EXAMPLES

Hereinafter, the present invention will be described with reference to Examples, but the present invention is not limited to the following Examples.

### Example 1

### (Binding of Antifreeze Protein to Target Protein Using Protein Crosslinking Agent)

By using a protein crosslinking agent to bind the antifreeze protein to an amino group on the surface of the target protein, an attempt was made to control the posture of the target protein with respect to the electron beam incidence direction.

### 1. Crosslinking Reaction of HPLC6 and Target Protein

As the target protein, a hemagglutinin (HA) protein (manufactured by MyBioSource, Inc., MBS434205) was used. As the antifreeze protein, an HPLC6 peptide (amino acid sequence: SEQ ID NO: 1) which is a type I antifreeze protein was expressed in Escherichia coli as a GST fusion protein, and then GST was cleaved and purified for use. The HA protein (concentration: 1 mg/mL) and the HPLC6 peptide (concentration: 1.7 mg/mL), which were each dissolved in PBS, were mixed at a molar ratio of 1:0, 1:4, 1:22, or 1:110, the mixture was subjected to a crosslinking reaction at room temperature (about 25 °C) for 5 minutes in the presence of 0.14% by mass of glutaraldehyde as a protein crosslinking agent, the crosslinking reaction was stopped by adding a 1 M Tris hydrochloride buffer solution (pH 8.0) thereto such that a final concentration was 44 mM, and then each sample solution (concentration of HA protein: 1.6 μM (the concentration of HA protein is the same in each sample, and the concentration of HPLC6 peptide is different)) was prepared.

### 2. Production of Sample Grid

After a hydrophilic treatment was performed on a film hole grid (Quantifoil R1.2/1.3 Cu 300) by glow discharge, 3 μL of the sample solution prepared in “1.” was placed on the grid. Next, the excess solution was removed with filter paper, and then the grid was rapidly dropped into liquid ethane to freeze the sample. Accordingly, a frozen grid in which the protein sample was trapped in a thin amorphous ice film was obtained. The obtained frozen grid was stored in liquid nitrogen until observation with a cryo-electron microscope.

3. Observation with Cryo-Electron Microscope

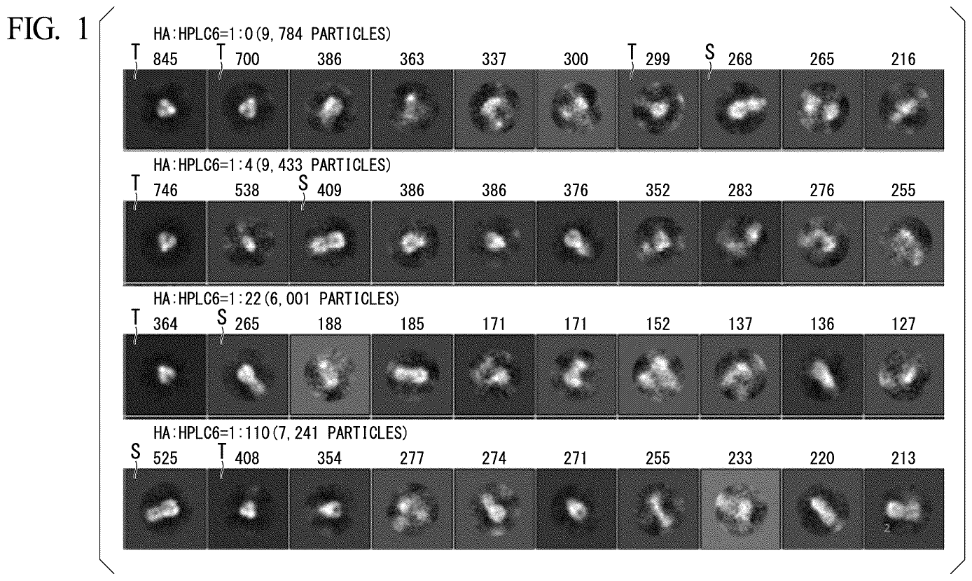

Each of the frozen grids produced in the section “2.” was inserted into a cryo-electron microscope (manufactured by Thermo Fisher Scientific, Inc., Talos Arctica), and observed and imaged at an acceleration voltage of 200 kV. From the captured image, using RELION, particle images projected in various orientations were cut out, classified into particle images in the same orientation, aligned, and then the orientations and positions of the classified particle images were aligned for each projection direction to obtain an average image. The results are shown in FIG. 1. In FIG. 1, the number described next to the molar ratio of the HA protein to the HPLC6 is the total number of projected particle images. In addition, the number described on the particle image indicates the number of particle images in the same orientation, and the particle images were aligned from left to right in order of the number of particle images. In addition, in FIG. 1, the reference numeral “T” indicates a particle image obtained by projecting the HA protein from above, and the reference numeral “S” indicates a particle image obtained by projecting the HA protein from the side.

As shown in FIG. 1, in the molar ratio of the HA protein to the HPLC6 peptide, as the mole fraction of the HPLC6 peptide increases, the number of particle images of the HA protein projected from the side tends to increase. In a case where the molar ratio of the HA protein to the HPLC6 peptide is 1:0 to 1:22, the number of particle images of the HA protein projected from above is the largest, but in a case where the molar ratio of the HA protein to the HPLC6 peptide is 1:110, the number of particle images of the HA protein projected from the side is the largest. It is presumed that, since as the mole fraction of the HPLC6 peptide increases, the amount of the HPLC6 peptide bonded to the lysine residue or the N-terminal amino group present on the side (side surface) of the HA protein increases, and the surface (side surface) to which the HPLC6 peptide is bonded faces the gas-liquid interface, the particle image of the HA protein projected from the side increases.

Therefore, it was confirmed that in the molar ratio of the target protein to the HPLC6 peptide, the posture of the target protein with respect to the electron beam incidence direction can be controlled by increasing the mole fraction of the HPLC6 peptide, whereby a large number of particle images projected in various directions can be collected.

### Example 2

(Effect of Suppressing Adsorption of Target Protein onto Amorphous Carbon Film by Antifreeze Protein)

It was examined that for a target protein having a property of easily adsorbing onto an amorphous carbon film in a grid, a fusion protein of an antifreeze protein and a target protein is prepared by genetic engineering, thereby suppressing the adsorption onto the amorphous carbon film and dispersing the target protein over the entire observation region.

### 1. Expression and Purification of Fusion Protein

As the target protein, glutamine synthetase was used. The glutamine synthetase is composed of a homododecamer, and has a hexagonal columnar shape with a diameter of about 16 nm and a height (thickness) of about 11 nm. The monomer of glutamine synthetase self-assembles in a solution to form a dodecamer. A nucleic acid encoding a fusion protein as the antifreeze protein, in which an HPLC6 peptide (amino acid sequence: SEQ ID NO: 1), which is a type I antifreeze protein, was bonded to an N-terminal of a monomer of glutamine synthetase (amino acid sequence: SEQ ID NO: 3) through a linker sequence (amino acid sequence: SEQ ID NO: 4, base sequence: SEQ ID NO: 5) was inserted into the plasmid vector pET-28a (manufactured by Novagen Inc.). A strain of Escherichia coli BL21 (DE3) was transformed with the vector. The target protein was purified from a soluble fraction of the induced Escherichia coli using nickel column, and after the His tag was cleaved, the target protein was further purified using a gel filtration column.

In addition, as a control, a nucleic acid (base sequence: SEQ ID NO: 6) encoding only a monomer of glutamine synthetase to which no antifreeze protein is bonded was inserted into a plasmid vector, and the expression and purification were performed in the same manner using an Escherichia coli expression system.

### 2. Production of Sample Grid

For the sample containing the glutamine synthetase to which the HPLC6 peptide was bonded, the sample was diluted with a buffer solution (composition: 20 mM Tris-HCl (pH 7.5), 100 mM NaCl, 5 mM MgCl₂) such that the protein concentration was 2.0 μM, and for the sample containing only the glutamine synthetase, the sample was diluted with the buffer solution such that the protein concentration was 2.0 μM, and the sample grid was prepared using the same method as that of “2.” of Example 1.

3. Observation with Cryo-Electron Microscope

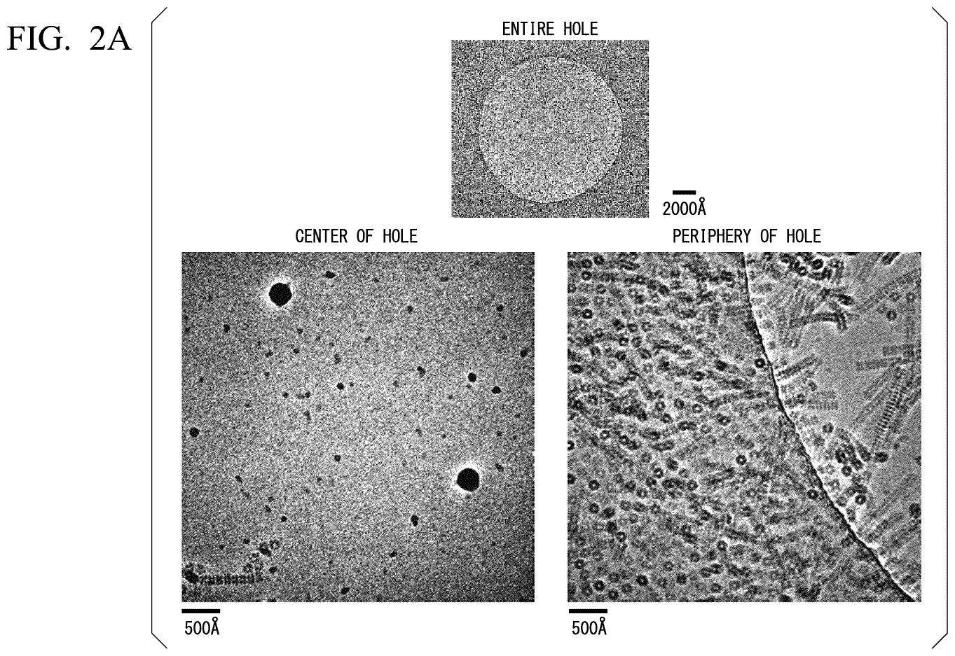

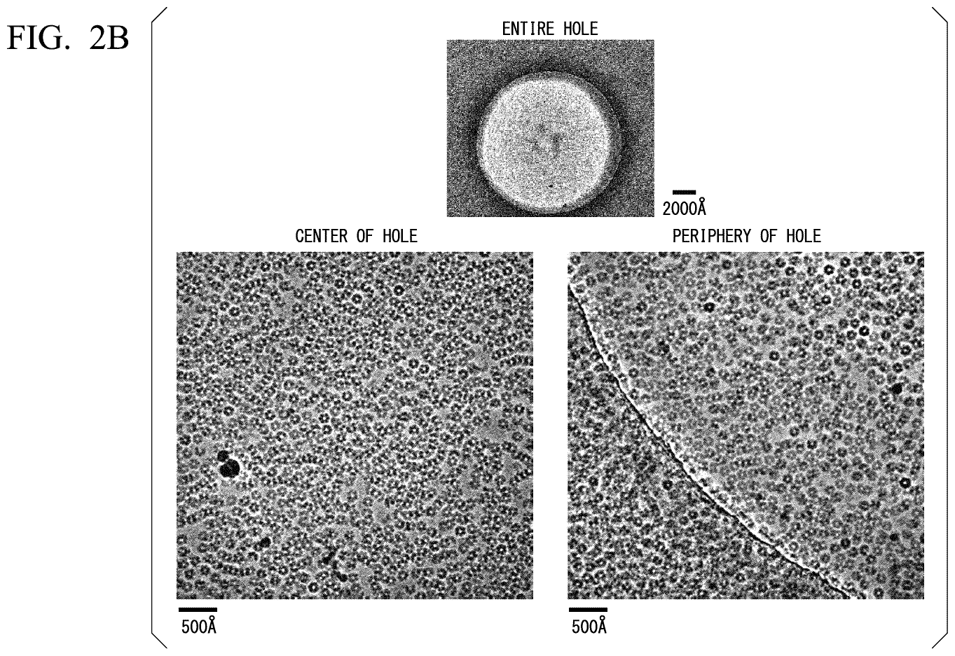

Each of the frozen grids produced in the section “2.” was inserted into a cryo-electron microscope (manufactured by Thermo Fisher Scientific, Inc., Talos Arctica), and observed and imaged at an acceleration voltage of 200 kV. The results are shown in FIG. 2A (only glutamine synthetase) and FIG. 2B (glutamine synthetase to which HPLC6 peptide is bonded).

As shown in FIG. 2A, in the sample grid containing only the glutamine synthetase, the glutamine synthetase was not observed in the center of the hole, and the glutamine synthetase was adsorbed onto the amorphous carbon film in the periphery of the hole and was not dispersed in the entire observation region.

On the other hand, as shown in FIG. 2B, in the sample grid containing the glutamine synthetase to which the HPLC6 peptide was bonded, particles were uniformly dispersed in both the center of the hole and the periphery of the hole.

In addition, in FIG. 2B, a particle image obtained by projecting a dodecamer of glutamine synthetase from above was mainly observed. It was presumed that, in the sample, the monomer of the glutamine synthetase to which the HPLC6 peptide was bonded at the N-terminal formed a dodecamer and the surface (upper surface) to which the HPLC6 peptide was bonded faced the gas-liquid interface, whereby the posture of the dodecamer of the glutamine synthetase with respect to the electron beam incidence direction was aligned and a particle image projected from above is mainly observed.

Furthermore, in the entire image of the hole, it was confirmed that the inside of the hole was whitish as a whole and a uniform and thin ice was formed.

Therefore, it was confirmed that a fusion protein with the antifreeze protein was formed for the target protein having a property of easily adsorbing onto the amorphous carbon film in the grid, whereby the adsorption onto the amorphous carbon film was suppressed, and the particles of the target protein can be dispersed over the entire observation region.

In addition, it was found that the thickness of the ice of the grid can be controlled to be thin and uniform by using a homo-multimeric protein in which the thickness and the particle diameter are uniform.

### Example 3

(Control of Thickness of Ice by Homo-Multimeric Protein to which Antifreeze Protein is Bonded)

It was examined whether the thickness of the ice of the grid can be controlled to be thin and uniform by forming the fusion protein with the antifreeze protein even for a homo-multimeric protein having a uniform thickness and particle diameter other than the glutamine synthetase.

### 1. Expression and Purification of Fusion Protein

As the target protein, ferritin was used. In Example 3, the ferritin serves as a role of a target protein and a role of an ice-thickness controlling molecule. Ferritin is composed of a homo-24-mer and has a spherical shape with a diameter of about 14 nm. The monomer of ferritin self-assembles in a solution to form a 24-mer. A nucleic acid encoding a fusion protein as the antifreeze protein, in which an HPLC6 peptide (amino acid sequence: SEQ ID NO: 1), which is a type I antifreeze protein, was bonded to an N-terminal of a monomer of ferritin (amino acid sequence: SEQ ID NO: 7) through a linker sequence (amino acid sequence: SEQ ID NO: 8, base sequence: SEQ ID NO: 9) was inserted into the plasmid vector pET-28a (manufactured by Novagen Inc.). Escherichia coli was transformed with the vector. The target protein was purified from a soluble fraction of the induced Escherichia coli using nickel column, and after the His tag was cleaved, the target protein was further purified using a gel filtration column.

In addition, as a control, a nucleic acid (base sequence: SEQ ID NO: 10) encoding only a monomer of ferritin to which no antifreeze protein is bonded was inserted into a plasmid vector, and the expression and purification were performed in the same manner using an Escherichia coli expression system.

### 2. Production of Sample Grid

For the sample containing the ferritin to which the HPLC6 peptide was bonded, the sample was diluted with a buffer solution (composition: 20 mM Tris-HCl (pH 7.5), 100 mM NaCl, 5 mM MgCl₂) such that the protein concentration was 2 μM, and for the sample containing only the ferritin, the sample was diluted with the buffer solution such that the protein concentration was 7 μM, and the sample grid was prepared using the same method as that of “2.” of Example 1.

3. Observation with Cryo-Electron Microscope

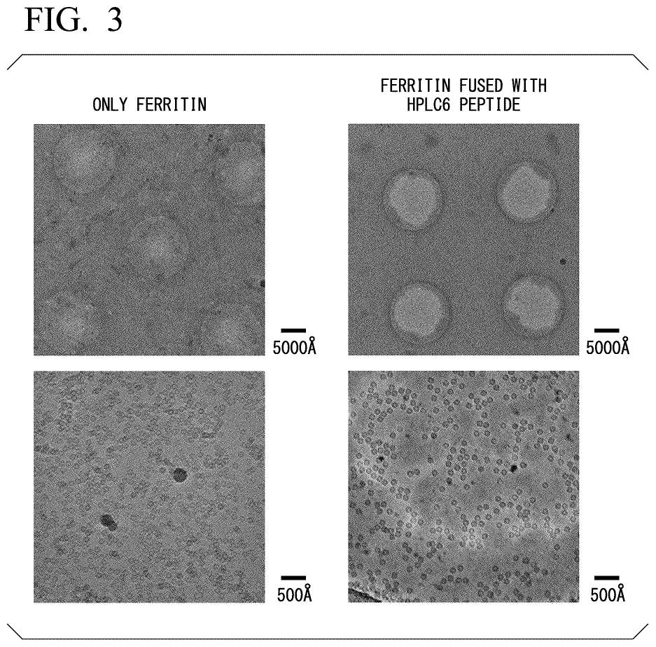

Each of the frozen grids produced in the section “2.” was inserted into a cryo-electron microscope (manufactured by Thermo Fisher Scientific, Inc., Talos Arctica), and observed and imaged at an acceleration voltage of 200 kV. The results are shown in FIG. 3 (left: only ferritin, right: ferritin to which HPLC6 peptide is bonded)

As shown in FIG. 3, in the sample grid containing only ferritin, only the central portion inside the hole was whitish, and ice was thin only in the central portion.

On the other hand, it was confirmed that in the sample grid containing the ferritin to which the HPLC6 peptide was bonded, the inside of the hole was overall whitish and a uniform and thin ice was formed. It was presumed that, in the sample, the monomers of ferritin to which the HPLC6 peptide was bonded at the N-terminal form a 24-mer, and the surface (upper surface) to which the HPLC6 peptide was bonded faced the gas-liquid interface and was dispersed over the entire hole, and as a result, the thickness of the ice was uniformly formed according to the diameter of the ferritin.

Therefore, it was confirmed that, regardless of the type of homo-multimeric protein, the thickness of the ice of the grid can be controlled to be thin and uniform by using a homo-multimeric protein in which the thickness and the particle diameter are uniform.

### Example 4

(Control of Ice Thickness and Dispersion Effect of Target Protein by Homo-Multimeric Protein to which Antifreeze Protein is Bonded)

It was examined that the target protein having a property of easily aggregating in the central portion of the observation region of the grid was mixed with the homo-multimeric protein to which the antifreeze protein was bonded, thereby suppressing the aggregation of the target protein in the central portion of the observation region of the grid while controlling the thickness of the ice of the grid to be thin and uniform, and dispersing the target protein over the entire observation region.

### 1. Expression and Purification of Fusion Protein

As the target protein, an HA protein (manufactured by MyBioSource, Inc., MBS434205) was used. As the fusion protein, a fusion protein as the antifreeze protein, in which the HPLC6 peptide, which is a type I antifreeze protein, was bonded to the N-terminal of the monomer of ferritin was expressed and purified by the same method as in “1.” of Example 3 described above.

### 2. Production of Sample Grid

For a sample containing ferritin with which the HA protein and the HPLC6 peptide were fused, the sample was diluted with a buffer solution (composition: 20 mM Tris-HCl (pH 7.5), 100 mM NaCl, 5 mM MgCl₂) such that the concentration of the HA protein was 3.0 μM and the concentration of the HPLC6 peptide was 1.0 μM, and the sample grid was prepared using the same method as that of “2.” in Example 1.

3. Observation with Cryo-Electron Microscope

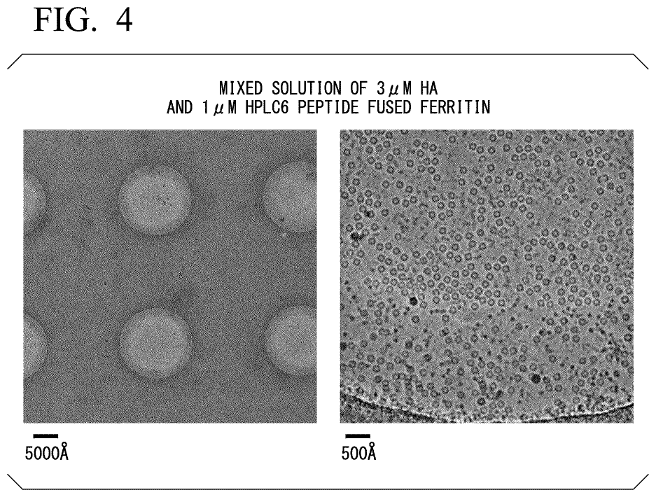

Each of the frozen grids produced in the section “2.” was inserted into a cryo-electron microscope (manufactured by Thermo Fisher Scientific, Inc., Talos Arctica), and observed and imaged at an acceleration voltage of 200 kV. The results are shown in FIG. 4.

As shown in the image on the left side of FIG. 4, it was confirmed that the inside of the hole was whitish as a whole and uniform and thin ice was formed. In addition, as shown in the image on the right side of FIG. 4, the HA protein was dispersed in the entire observation region.

Therefore, it was confirmed that the target protein can be dispersed over the entire observation region while controlling the thickness of the ice of the grid to be thin and uniform by using the target protein and the homo-multimeric protein to which the antifreeze protein was bonded, in combination.

### Example 5

(Suppression of Bias of Posture by Effect of Suppressing Adsorption of Target Protein onto Gas-Liquid Interface by Protecting Gas-Liquid Interface of Antifreeze Protein)

It was examined to suppress the bias of the posture of the target protein by mixing the target protein having a property of easily adsorbing onto the gas-liquid interface in a specific posture with the antifreeze protein to protect the gas-liquid interface and suppress the target protein from colliding with the gas-liquid interface and adsorbing thereonto.

### 1. Preparation of Target Protein and Antifreeze Protein

As the target protein, a mutant Y12A of inosine-5′-monophosphate dehydrogenase 2 (IPDH2) (hereinafter, referred to as “IMPDH2 (Y12A)” in some cases) was used. IMPDH2 (Y12A) was prepared by introducing a Y12A mutation into a plasmid containing a gene encoding wild-type IMPDH2, and then expressing IMPDH2 (Y12A) in Escherichia coli. As the antifreeze protein, an HPLC6 peptide (amino acid sequence: SEQ ID NO: 1) which is a type I antifreeze protein was expressed in Escherichia coli as a GST fusion protein, and then GST was cleaved and purified for use.

### 2. Production of Sample Grid

For the sample containing IMPDH2 (Y12A) and HPLC6 peptide, the sample was diluted with a buffer solution (composition: 20 mM Tris-HCl (pH 7.5), 100 mM NaCl, 5 mM MgCl₂) such that the molar ratio of IMPDH2 (Y12A) to HPLC6 peptide was 1:1.25, and the sample grid was prepared using the same method as that of “2.” in Example 1. As a grid, a film hole grid (Quantifoil R1.2/1.3 Cu 300) and a graphene grid (manufactured by AirMembrane Co., Ltd., 2-layer graphene TEM grid Quantifoil R1.2/1.3 300 mesh Au hydrophilized) were used. As a control, a sample containing only the target protein was also prepared.

3. Observation with Cryo-Electron Microscope

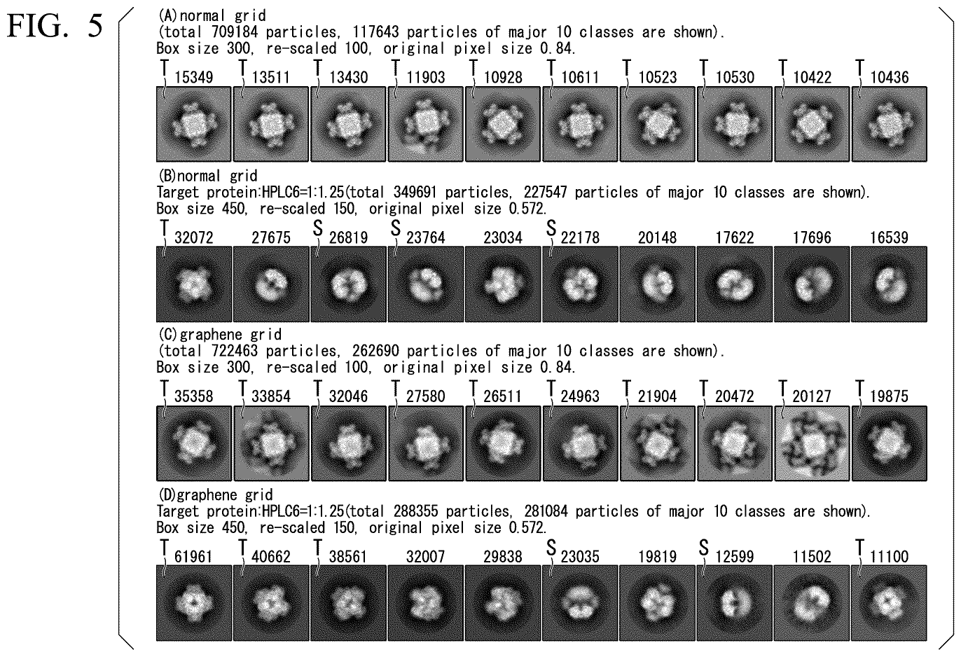

Each of the frozen grids produced in the section “2.” was inserted into a cryo-electron microscope (manufactured by Thermo Fisher Scientific, Inc., Talos Arctica), and observed and imaged at an acceleration voltage of 200 kV. The results are shown in FIG. 5. In FIG. 5, the reference numeral “T” indicates a particle image obtained by projecting IMPDH2 (Y12A) from above, and the reference numeral “S” indicates a particle image obtained by projecting IMPDH2 (Y12A) from the side.

As shown in (A) and (C) of FIG. 5, in a sample containing only IMPDH2 (Y12A), which is a target protein, IMPDH2 (Y12A) was adsorbed onto a gas-liquid interface in a specific posture, and the particle images in which IMPDH2 (Y12A) was projected from above were accounted for the majority. On the other hand, as shown in (B) and (D) of FIG. 5, in the sample containing IPDH2 (Y12A) and HPLC6 peptide, a particle image in which IMPDH2 (Y12A) was projected from the side or from obliquely above was also observed.

Therefore, it was confirmed that in a case where the target protein and the HPLC6 peptide were mixed and used, the target protein was suppressed from colliding with the gas-liquid interface and adsorbing thereonto, and a large number of particle images projected in various directions could be collected.

### Example 6

(Effect of Suppressing Adsorption of Target Protein onto Amorphous Carbon Film by Amphipathic Protein Other than Antifreeze Protein)

It was examined that for a target protein having a property of easily adsorbing onto an amorphous carbon film in a grid, a fusion protein of an amphipathic protein other than the antifreeze protein, and a target protein is prepared, thereby suppressing the adsorption onto the amorphous carbon film and dispersing the target protein over the entire observation region.

### 1. Expression and Purification of Fusion Protein

As the target protein, ferritin was used. A nucleic acid encoding a fusion protein in which a protein consisting of an amino acid sequence (SEQ ID NO: 11) containing a KALA peptide as an example of an amphipathic protein other than the antifreeze protein was bonded to the N-terminal of a monomer of ferritin was inserted into the plasmid vector pET-28a (manufactured by Novagen Inc.). In this fusion protein, a monomer of ferritin and a protein containing a KALA peptide are linked through a linker sequence (amino acid sequence: SEQ ID NO: 8). A strain of Escherichia coli BL21 (DE3) was transformed with the vector. The target protein was purified from a soluble fraction of the induced Escherichia coli using nickel column, and after the His tag was cleaved, the target protein was further purified using a gel filtration column.

In addition, as a control, a sample containing only ferritin was also prepared using the Escherichia coli expression system without the sequence of the amphipathic protein, as described in detail in “1.” of Example 3.

### 2. Production of Sample Grid

For the sample containing ferritin to which a KALA peptide was bonded and the sample containing only ferritin, the samples were each diluted with a buffer solution (composition: 20 mM Tris-HCl (pH 7.5), 100 mM NaCl, 5 mM MgCl₂) such that the protein concentration was 2.0 μM, and the frozen grid of the sample was prepared using the same method as in “2.” of Example 1.

3. Observation with Cryo-Electron Microscope

Each of the frozen grids produced in the section “2.” was inserted into a cryo-electron microscope (manufactured by Thermo Fisher Scientific, Inc., Talos Arctica), and observed and imaged at an acceleration voltage of 200 kV.

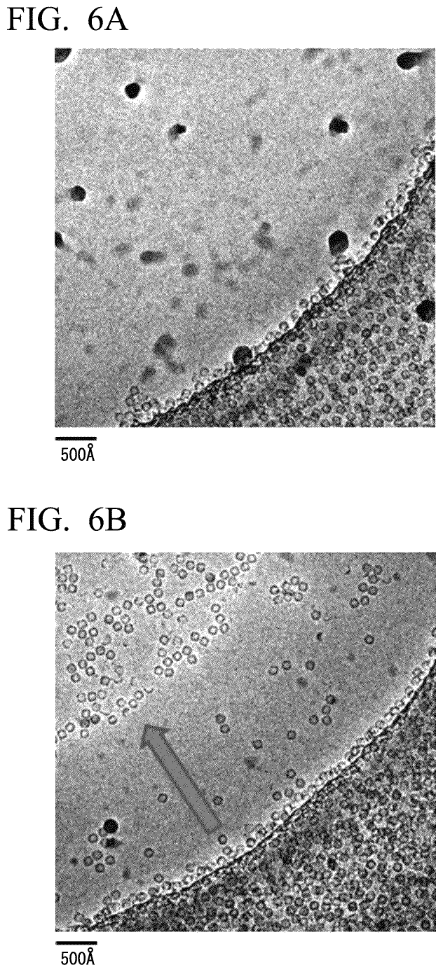

FIG. 6A is an image of ferritin captured by a cryo-electron microscope in Example 6, and FIG. 6B is an image of a fusion protein of ferritin and a KALA peptide captured by a cryo-electron microscope in Example 6.

As shown in FIG. 6A, in the frozen grid containing only ferritin, the ferritin was not observed in the center of the hole, the ferritin was adsorbed onto the amorphous carbon film in the periphery of the hole, and the ferritin was not dispersed over the entire observation region.

In contrast, as shown in FIG. 6B, in the frozen grid containing the ferritin to which the KALA peptide was bonded, the particles of the fusion protein were uniformly dispersed in both the center of the hole indicated by the arrow and the periphery of the hole.

From the above results, it was confirmed that even in the case of an amphipathic protein other than the antifreeze protein, it is possible to effectively suppress the adsorption of the target protein onto the amorphous carbon film in the periphery of the hole. It is considered that the present embodiment can be applied to proteins other than HPLC6, for example, a protein having another amphipathic α-helix structure, other antifreeze proteins, and other amphipathic proteins.

### INDUSTRIAL APPLICABILITY

According to the cryo-electron microscope sample of the present embodiment, it is possible to obtain a cryo-electron microscope sample, in which a bias of an undesirable posture of the target protein in a sample with respect to an electron beam incidence direction and a collision with and adsorption onto a gas-liquid interface are suppressed, and in which the target protein is dispersed over an entire observation region. In addition, according to the cryo-electron microscope sample of the present embodiment, it is possible to obtain a cryo-electron microscope sample in which the thickness of ice of a sample is uniformly controlled.

### REFERENCE SIGNS LIST

S: particle image obtained by projecting protein from the side
T: particle image obtained by projecting protein from above

1. A cryo-electron microscope sample preparation kit, comprising:
an amphipathic protein or an expression vector containing a nucleic acid encoding the amphipathic protein.

2. A cryo-electron microscope sample preparation kit, comprising:
an ice-thickness controlling molecule to which an amphipathic protein is bonded.

3. The cryo-electron microscope sample preparation kit according to claim 1,
wherein the amphipathic protein has an amphipathic α-helix structure.

4. The cryo-electron microscope sample preparation kit according to claim 1,
wherein the amphipathic protein is an antifreeze protein.

5. The cryo-electron microscope sample preparation kit according to claim 4,
wherein the antifreeze protein is a type I antifreeze protein.

6. The cryo-electron microscope sample preparation kit according to claim 5,
wherein the type I antifreeze protein is an HPLC6 peptide.

7. The cryo-electron microscope sample preparation kit according to claim 1,
wherein the amphipathic protein is a KALA peptide.

8. The cryo-electron microscope sample preparation kit according to claim 1, further comprising:
a protein crosslinking agent.

9. The cryo-electron microscope sample preparation kit according to claim 8,
wherein the protein crosslinking agent is an amine-reactive crosslinking agent.

10. The cryo-electron microscope sample preparation kit according to claim 9,
wherein the amine-reactive crosslinking agent is glutaraldehyde, or bis(3-sulfo-N-succinimidyl)suberate or a salt thereof.

11. The cryo-electron microscope sample preparation kit according to claim 1, further comprising:
an ice-thickness controlling molecule to which an amphipathic protein is bonded.

12. The cryo-electron microscope sample preparation kit according to claim 2,
wherein the ice-thickness controlling molecule is an antifreeze protein and is a homo-multimeric protein.

13. The cryo-electron microscope sample preparation kit according to claim 12,
wherein the homo-multimeric protein is a glutamine synthetase or ferritin.

14. A preparation method for a cryo-electron microscope sample, comprising:
preparing a cryo-electron microscope sample containing a target protein using the cryo-electron microscope sample preparation kit according to claim 1.

15. A method for controlling the posture of a target protein in a cryo-electron microscope sample, the method comprising:
preparing a cryo-electron microscope sample containing a target protein using the cryo-electron microscope sample preparation kit according to claim 1.

16. A method for suppressing adsorption of a target protein in a cryo-electron microscope sample onto an amorphous carbon film, the method comprising:
preparing a cryo-electron microscope sample containing a target protein using the cryo-electron microscope sample preparation kit according to claim 1.

17. A method for suppressing collision and adsorption of a target protein in a cryo-electron microscope sample onto a gas-liquid interface, the method comprising:
preparing a cryo-electron microscope sample containing a target protein using the cryo-electron microscope sample preparation kit according to claim 1.

18. A method for controlling a thickness of ice in a cryo-electron microscope sample, the method comprising:
preparing a cryo-electron microscope sample containing a target protein using the cryo-electron microscope sample preparation kit according to claim 2.

19. The cryo-electron microscope sample preparation kit according to claim 1, further comprising:
a film hole grid.
